(19) United States
(12) Patent Application Publication
(10) Pub. No.: US 2026/0233007 A1
(43) Pub. Date: Aug. 13, 2026

(54) AUTOMATICALLY CALCULATED DEEP BRAIN STIMULATION PROGRAMMING SETTINGS BASED ON PATIENT-SPECIFIC ANATOMY

(71) Applicant: MEDTRONIC, INC., Minneapolis, MN (US)

(72) Inventors: Jeffrey J. Zweber, Blaine, MN (US); Jerel K. Mueller, Saint Paul, MN (US); Lukas J. Valine, Minneapolis, MN (US)

(21) Appl. No.: 19/149,804

(22) PCT Filed: Jan. 18, 2024
(86) PCT No.: PCT/IB2024/050491

§ 371 Date: Jul. 21, 2025

Related U.S. Application Data
(60) Provisional application No. 63/441,289, filed on Jan. 26, 2023.

(51) Int. Cl. A61N 1/36; A61N 1/05

(52) U.S. Cl. CPC A61N 1/36132; A61N 1/0534; A61N 1/36175

(57) ABSTRACT

A system for therapy program selection for a patient having an implanted medical device can comprise a programmer device configured to receive patient-specific anatomy data including a location and an orientation for an implanted lead relative to one or more anatomical structures, wherein the one or more anatomical structures include a target structure, receive an indication of stimulation field criteria, determine stimulation settings based on the patient-specific anatomy data and the indication, and display, via a user interface of the programmer device, a visualization of a VNA produced by the determined stimulation settings within the patient-specific anatomy data.

### FIELD

This Application claims priority from U.S. Provisional Patent Application 63/441,289, filed 26 Jan. 2023, the entire content of which is incorporated herein by reference.

The present technology is generally related to deep brain stimulation programming settings and more particularly to automatically calculated selections of Volume of Neural Activation (VNA) based on patient-specific anatomy.

### BACKGROUND

Implantable medical devices, such as electrical stimulators or therapeutic agent delivery devices, have been proposed for use in different therapeutic applications, including deep brain stimulation (DBS). In some therapy systems, an implantable electrical stimulator delivers electrical therapy to a target tissue site within a patient with the aid of one or more electrodes, which may be deployed by medical leads and/or on a housing of the electrical stimulator, or both. In some therapy systems, therapy may be delivered via particular combinations of the electrodes carried by leads and/or by the housing of the electrical stimulator.

During a programming session, which may occur during implant of the medical device, during a trial session, or during an in-clinic or remote follow-up session after the medical device is implanted in the patient, a clinician may generate one or more therapy programs (also referred to as therapy parameter sets) that are found to provide efficacious therapy to the patient, where each therapy program may define values for a set of therapy parameters. A medical device may deliver therapy to a patient according to one or more stored therapy programs. In the case of electrical stimulation, the therapy parameters may define characteristics of the electrical stimulation waveform to be delivered. In examples in which electrical stimulation is delivered in the form of electrical pulses, for example, the therapy parameters may include an electrode configuration including an electrode combination and electrode polarities, a stimulation amplitude, which may be a current or voltage amplitude, a pulse width, and a pulse rate. The electrode configuration ultimately produces a Volume of Neural Activation (VNA) or volume that is stimulated when the electrical stimulation waveform is delivered.

Modern DBS systems offer increasing flexibility in programming to allow the programming clinician to attempt to stimulate as much of the anatomical target (structure) in the brain as feasible. There are times where stimulation of adjacent structures may cause side effects, in which case the programming can be adjusted to avoid those structures. The structures in question are rarely uniform and symmetric, so the programmers allow for programming the stimulation field in asymmetric shapes. With this amount of programming flexibility comes increase complexity, which can lead to frustration and/or increased time in the operating room or in the clinic programming the DBS system. Because there are so many degrees of freedom and flexibility with programming, it can be unclear where to start, so in the interest of time and to reduce complexity, many physicians simply start with a very basic symmetric stimulation field shape. This shape rarely matches the best shape fit for the structure of interest and can spill over onto adjacent undesirable structures that can produce unwanted side effects. From that starting stimulation field shape clinicians may have to continue to adjust the shape until a desired clinical outcome with the least side effects is achieved. Selection of effective stimulation parameters for DBS therapy can therefore be time-consuming (e.g., longer and more frequent medical visits) for both the clinician and the patient. Further, even following this trial-and-error approach the outcome may still not be the best that could have been achieved and/or the side effects at their lowest.

Accordingly, there is a need to simplify the DBS programming experience for clinicians while improving efficiency of arriving at optimal stimulation field shapes.

### SUMMARY

The techniques of this disclosure generally relate to simplifying the clinician experience by automatically presenting a clinician with one or more pre-defined selections that correspond to commonly desired stimulation field criteria and automatically generating a Volume of Neural Activation (VNA) based on the selected criteria.

In one aspect, the present disclosure provides a system for therapy program selection for a patient having an implanted medical device. The system comprises a programmer device including computing hardware of at least one processor and memory operably coupled to the at least one processor and instructions. When executed on the programmer device the instructions cause the programmer device to receive patient-specific anatomy data including a location and an orientation for an implanted lead relative to one or more anatomical structures, wherein the one or more anatomical structures include a target structure, receive an indication of stimulation field criteria, determine stimulation settings based on the patient-specific anatomy data and display, via a user interface of the programmer device, a visualization of a VNA produced by the determined stimulation settings within the patient-specific anatomy data.

In another aspect, the disclosure provides a method for therapy program selection for a patient having an implanted medical device. The method comprises receiving patient-specific anatomy data including a location and an orientation for an implanted lead relative to one or more anatomical structures, wherein the one or more anatomical structures include a target structure, receiving an indication of stimulation field criteria, determining stimulation settings based on the patient-specific anatomy data, and displaying a visualization of a VNA produced by the determined stimulation settings within the patient-specific anatomy data.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

FIG. 1A is a schematic illustrating an example deep brain stimulation (DBS) system configured to deliver electrical stimulation therapy to a tissue site within a brain of a patient, according to an embodiment.

FIG. 1B is a block diagram illustrating components of the system of FIG. 1A, according to an embodiment.

FIG. 2 is a block diagram of a system configured to deliver electrical stimulation therapy to a tissue site within a brain of a patient, according to an embodiment.

FIG. 3 is a flowchart of a method for automatically calculating a VNA based on user selected criteria according to an embodiment.

FIG. 4 is an example electrode visualization for a lead in an anatomical target, according to an embodiment.

FIG. 5 is an example user interface for visualizing a lead in an anatomical target, according to an embodiment.

While various embodiments are amenable to various modifications and alternative forms, specifics thereof have been shown by way of example in the drawings and will be described in detail. It should be understood, however, that the intention is not to limit the claimed inventions to the particular embodiments described. On the contrary, the intention is to cover all modifications, equivalents, and alternatives falling within the spirit and scope of the subject matter as defined by the claims.

### DETAILED DESCRIPTION

Embodiments of the present disclosure simplify the clinician experience by automatically presenting a clinician with one or more pre-set selections that correspond to desired stimulation field criteria. Stimulation field criteria can include one or more of maximum Volume of Neural Activation (VNA) coverage of a structure, maximum VNA coverage of a structure with no VNA outside of the structure, maximum VNA of a structure with the clinician specifying one or more structures to have no VNA, and other multi-objective optimizations between structures to be stimulated and avoided.

The one or more pre-set selections can be presented via a clinical programmer that has access to patient-specific brain anatomy. Once a selection has been made, the programmer can determine the set of stimulation settings that would meet the selected stimulation criteria. The VNA (Volume of Neural Activation) displayed in relation to the anatomy and what stimulation settings produced this VNA is then shown to the clinician. The clinician then would have the option to use this as the starting point for either adjusting the VNA based on their clinical judgement or using the suggested settings as-is before beginning stimulation.

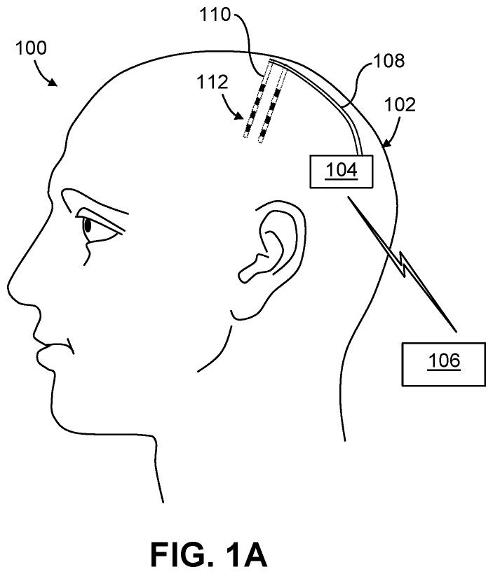

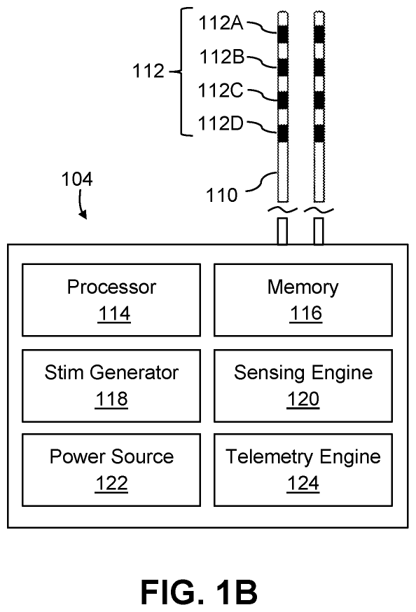

Referring to FIG. 1A, an example deep brain stimulation (DBS) system 100 configured to deliver electrical stimulation therapy to a tissue site within a brain of a patient 102 is depicted, according to an embodiment. In the example shown in FIG. 1A, therapy system 100 includes medical device programmer 106, implantable medical device (IMD) 104, lead extension 108, and one or more leads 110 with respective sets of electrodes 112. FIG. 1B is a block diagram better illustrating certain components of system 100, particularly components of IMD 104.

IMD 104 generally includes a processor 114, memory 116, a stimulation generator 118, a sensing engine 120, a power source 122, and a telemetry engine 124.

Processor 114 can include one or more digital signal processors (DSPs), general purpose microprocessors, application specific integrated circuits (ASICs), field programmable logic arrays (FPGAs), or other equivalent integrated or discrete logic circuitry, or combinations thereof. The functions attributed to processors described herein may be provided by a hardware device and embodied as software, firmware, hardware, or any combination thereof. Processor 114 is configured to control stimulation generator 118 according to therapy programs stored by memory 116 to apply particular stimulation parameter values specified by one or more programs, such as amplitude, pulse width, and pulse rate.

Memory 116 can be operably coupled to processor 114 and can include any volatile or non-volatile media, such as a random access memory (RAM), read only memory (ROM), non-volatile RAM (NVRAM), electrically erasable programmable ROM (EEPROM), flash memory, and the like. Memory 116 can store computer-readable instructions that, when executed by processor 114, cause IMD 104 to perform various functions described herein.

In an embodiment, memory 116 can store therapy programs (also referred to herein as “a set of stimulation settings”), operating instructions, and the like. Each stored therapy program defines a particular program of therapy in terms of respective values for electrical stimulation parameters. Operating instructions guide general operation of IMD 104 under control of processor 114 and can include instructions for monitoring brain signals within one or more brain regions via electrodes 112 and delivering electrical stimulation therapy to patient 102.

Stimulation generator 118 is configured to generate and deliver electrical stimulation therapy to one or more regions of brain of patient 102 via one or more electrodes 112 of one or more leads 110, respectively. IMD 104 is configured to deliver electrical stimulation therapy to the brain of patient 102 via stimulation generator 118 according to one or more stimulation therapy programs. A stimulation therapy program may define one or more electrical stimulation parameter values for therapy generated by stimulation generator 118 and delivered from IMD 104 to a target therapy delivery site within patient 102 via one or more electrodes 112. The electrical stimulation parameters may define an aspect of the electrical stimulation therapy, and may include, for example, voltage or current amplitude of an electrical stimulation signal, a charge level of an electrical stimulation, a frequency of the electrical stimulation signal, waveform shape, on/off cycling state (e.g., if cycling is “off,” stimulation is always on, and if cycling is “on,” stimulation is cycled on and off) and, in the case of electrical stimulation pulses, pulse rate, pulse width, and other appropriate parameters such as duration or duty cycle. In addition, if different electrodes are available for delivery of stimulation, a therapy parameter of a therapy program can be further characterized by an electrode combination, which can define selected electrodes 112 and their respective polarities. In some examples, stimulation may be delivered using a continuous waveform and the stimulation parameters can define this waveform.

In addition to being configured to deliver therapy to manage a disorder of patient 102, system 100 can be configured to sense bioelectrical brain signals or another physiological parameter of patient 102. For example, sensing engine 120 is configured to sense bioelectrical brain signals within one or more regions of the brain via electrodes 112. Accordingly, in some examples, electrodes 112 can be used to deliver electrical stimulation to target sites within the brain as well as sense brain signals. However, IMD 104 can also use a separate set of sensing electrodes to sense the bioelectrical brain signals. In some examples, the sensing engine of IMD 104 can sense bioelectrical brain signals via one or more of the electrodes 112 that are also used to deliver electrical stimulation to the brain. In other examples, one or more of electrodes 112 can be used to sense bioelectrical brain signals while one or more different electrodes 112 can be used to deliver electrical stimulation.

Power source 122 delivers operating power to various components of IMD 104. Power source 122 can include a small rechargeable or non-rechargeable battery and a power generation circuit to produce the operating power. Recharging can be accomplished through proximal inductive interaction between an external charger and an inductive charging coil within IMD 104. In some examples, power requirements may be small enough to allow IMD 104 to utilize patient motion and implement a kinetic energy-scavenging device to trickle charge a rechargeable battery. In other examples, traditional batteries may be used for a limited period of time.

Telemetry engine 124 is configured to support wireless communication between IMD 104 and external programmer 106 or another computing device under the control of processor 114. Processor 114 can receive, as updates to programs, values for various stimulation parameters such as amplitude and electrode combination, from programmer 106 via telemetry engine 124.

External programmer 106 is configured to wirelessly communicate with IMD 104 as needed to provide or retrieve therapy information. Programmer 106 is an external computing device that the user, e.g., the clinician and/or patient 102, can use to communicate with IMD 104. For example, programmer 106 can be a clinician programmer that the clinician uses to communicate with IMD 104 and program one or more therapy programs for IMD 104. In addition, or instead, programmer 106 can be a patient programmer that allows patient 102 to select programs and/or view and modify therapy parameter values. The clinician programmer can include more programming features than the patient programmer. In other words, more complex or sensitive tasks may only be allowed by the clinician programmer to prevent an untrained patient from making undesired changes to IMD 104.

Programmer 106 can be a hand-held computing device with a display viewable by the user and an interface for providing input to programmer 14 (i.e., a user input mechanism). For example, programmer 106 can include a small display screen (e.g., a liquid crystal display (LCD) or a light emitting diode (LED) display) that presents information to the user. In addition, programmer 106 can include a touch screen display, a keypad, buttons, a peripheral pointing device, voice activation, or another input mechanism that allows the user to navigate through the user interface (UI) of programmer 106 and provide input. If programmer 106 includes buttons and a keypad, the buttons may be dedicated to performing a certain function, e.g., a power button, and/or the buttons and the keypad may be soft keys that change in function depending upon the section of the UI currently viewed by the user, or any combination thereof.

In other examples, programmer 106 can be a larger workstation or a separate application within another multi-function device, rather than a dedicated computing device. For example, the multi-function device may be a notebook computer, tablet computer, workstation, one or more servers, cellular phone, personal digital assistant, or another computing device that may run an application that enables the computing device to operate as a secure medical device programmer 106. A wireless adapter coupled to the computing device can enable secure communication between the computing device and IMD 104.

When programmer 106 is configured for use by the clinician, programmer 106 may be used to transmit programming information to IMD 104. Programming information can include, for example, hardware information, such as the type of leads 110, the arrangement of electrodes 112 on leads 110, the position of leads 110 within the brain, one or more therapy programs defining therapy parameter values, therapeutic windows for one or more electrodes 112, and any other information that may be useful for programming into IMD 104. Programmer 106 can also be capable of completing functional tests (e.g., measuring the impedance of electrodes 112 of leads 110).

The clinician can also generate and store therapy programs within IMD 104 with the aid of programmer 106. Programmer 106 can assist the clinician in the creation/identification of therapy programs by providing a system for identifying potentially beneficial therapy parameter values. For example, during a programming session, programmer 106 may automatically suggest a combination of electrodes to the clinician for therapy delivery to the patient.

As depicted, system 100 can further comprise electrodes 112 of lead 110 that includes electrodes 112A-112D. Processor 114 can apply the stimulation signals generated by stimulation generator 118 to a selected combination of electrodes 112A-112D.

DBS can be used to treat or manage various patient conditions, such as, but not limited to, seizure disorders (e.g., epilepsy), pain, migraine headaches, psychiatric disorders (e.g., major depressive disorder (MDD), bipolar disorder, anxiety disorders, post-traumatic stress disorder, dysthymic disorder, and obsessive compulsive disorder (OCD)), behavior disorders, mood disorders, memory disorders, mentation disorders, movement disorders (e.g., essential tremor or Parkinson's disease), Huntington's disease, Alzheimer's disease, or other neurological or psychiatric disorders and impairment of patient 102.

Therapy systems configured for treatment of other patient conditions via delivery of therapy to the brain or another suitable target therapy delivery site in patient 102 can also be used in accordance with the techniques disclosed herein. For example, in other applications of system 100, the target therapy delivery site within patient 102 can be a location proximate to a spinal cord or to sacral nerves (e.g., the S2, S3 or S4 sacral nerves) in patient 102 or any other suitable nerve, organ, muscle or muscle group in patient 102, which can be selected based on, for example, a patient condition. For example, system 100 can be used to deliver electrical stimulation or a therapeutic agent to tissue proximate to a pudendal nerve, a perineal nerve, or other areas of the nervous system, in which cases, leads 110 are implanted and substantially fixed proximate to the respective nerve. As further examples, an electrical stimulation system may be positioned to deliver a stimulation to help manage peripheral neuropathy or post-operative pain mitigation, ilioinguinal nerve stimulation, intercostal nerve stimulation, gastric stimulation for the treatment of gastric mobility disorders and obesity, urinary dysfunction, fecal dysfunction, sexual dysfunction, muscle stimulation, for mitigation of other peripheral and localized pain (e.g., leg pain or back pain).

Leads 110 can be positioned to deliver electrical stimulation therapy to one or more target tissue sites within the brain to manage patient symptoms associated with a disorder of patient 102. Leads 110 may be implanted to position electrodes 112 at desired locations of the brain via any suitable technique, such as through respective burr holes in the skull of patient 102 or through a common burr hole in the cranium. Leads 110 can be placed at any location within the brain such that electrodes 112 are capable of providing electrical stimulation to target therapy delivery sites within the brain during treatment. Different neurological, motor, or psychiatric disorders can be associated with activity in one or more of regions of the brain, which may differ between patients. Accordingly, the target therapy delivery site for electrical stimulation therapy delivered by leads 110 may be selected based on the patient condition. For example, a suitable target therapy delivery site within the brain for controlling a movement disorder of patient 102 may include one or more of the pedunculopontine nucleus (PPN), thalamus, basal ganglia structures (e.g., globus pallidus, substantia nigra or subthalamic nucleus (STN)), zona inserta, fiber tracts, lenticular fasciculus (and branches thereof), ansa lenticularis, or the Field of Forel (thalamic fasciculus). The PPN may also be referred to as the pedunculopontine tegmental nucleus.

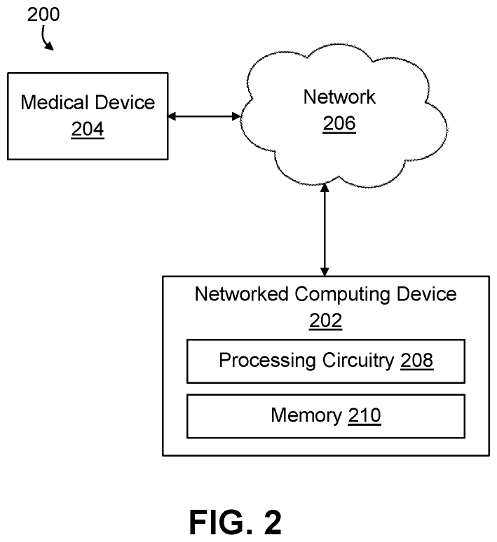

Referring to FIG. 2, a block diagram of a system 200 configured to deliver electrical stimulation therapy to a tissue site within a brain of a patient is depicted, according to an embodiment. System 200 includes networked computing device 202, medical device 204, and network 206.

For ease of explanation, networked computing device 202 is labeled separately from previously described programmer 106 and medical device 204 is labeled separately from previously described IMD 104, but one of ordinary skill in the art will readily understand that networked computing device 202 can be substantially similar to programmer 106 whereas medical device 204 can be substantially similar to IMD 104 as depicted and described in FIGS. 1A-1B.

Networked computing device 202 generally comprises processing circuitry 208 and memory 210. Of course, one of skill in the art will appreciate that networked computing device 202 can further comprise communication circuitry, a UI, and a power source (not shown). Processing circuitry 208 can include one or more processors that are configured to implement functionality and/or process instructions for execution within networked computing device 202. Memory 210 can be configured to store information within networked computing device 202 during operation. Memory 210 can include a computer-readable storage medium or computer-readable storage device.

In embodiments, the UI presented by networked computing device 206 can include a button or keypad, lights, a speaker for voice commands, a display, such as a liquid crystal (LCD), light-emitting diode (LED), or organic light-emitting diode (OLED). In some examples the display may be a touch screen. The UI is configured to display information related to the delivery of stimulation therapy, sensed patient signals, patient-specific anatomy, or any other such information. The UI can receive user input such as by a user pressing a button on a keypad or selecting an icon from a touch screen.

Network 206 comprises a communication network for connecting networked computing device 202 with medical device 204 (e.g., a wireless communication network, a wired communication network, a cellular communication network, the Internet, a short-range radio network (e.g., via Bluetooth)).

With reference to networked computing device 202 and network 206, embodiments of and the corresponding methods of configuring and operating system 200 can be performed in cloud computing, client-server, or other networked environment, or any combination thereof. The components of the system can be located in a singular “cloud” or network, or spread among many clouds or networks. End-user knowledge of the physical location and configuration of components of the system is not required.

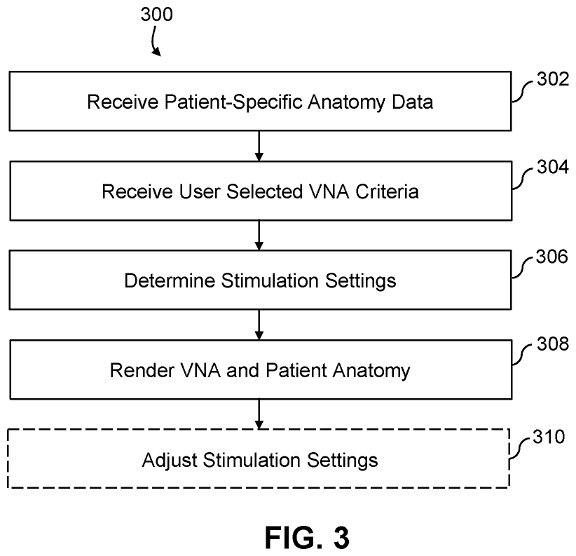

Referring to FIG. 3, a flowchart of a method 300 for automatically determining stimulation settings based on selected VNA criteria is depicted according to an embodiment. In embodiments method 300 can be implemented via a DBS system such as system 100 or system 200.

At 302, the programmer can receive patient-specific anatomy data. This anatomy can be oriented in relation to the implanted stimulation leads.

In some examples, patient-specific anatomy data can be received from a software application such as Sure Tune™ developed by Medtronic, Inc., of Minneapolis, Minnesota. SureTune™ is a therapy planning platform that enables the creation of patient-specific anatomy and lead location and orientation which then can be pulled into the DBS programmer for a visually informed programming session. In embodiments, a user may be able to toggle between various rendering views received from the software application, such as by toggling between a cylindrical mesh adjustment technique and a marching cubes technique.

Patient-specific anatomical data can include one or more anatomical targets. Anatomical targets can be tissue and/or structures to be stimulated via one or more electrodes. Stimulation of the specific areas and regions within an anatomical target can be desired for a patient-specific therapy program. For example, targeted stimulation can improve a patient dominant sub-symptom of Parkinson's disease, such as rigidity, bradykinesia, and tremors. Additionally, received anatomical data can include tissue and/or structures to be exempted from stimulation because stimulation via one or more electrodes may cause side-effects.

At 304, selectable settings are available for the programming clinician to choose. The selectable settings can be presented via the clinical programmer and be based on stimulation field criteria. These settings can be presented and selected via a UI displayed on the programmer or other computing device communicatively coupled to the medical device. A clinician can accordingly select the most appropriate stimulation field criteria via the programmer.

Stimulation field criteria can include one or more of maximum VNA coverage of a structure, maximum VNA coverage of a structure with no VNA outside of the structure, Maximum VNA of a structure with the clinician specifying one or more structures to have no VNA, and other multi-objective optimizations between structures to be stimulated and avoided. In embodiments, VNA coverage of a structure can be partial such that a desired portion of the structure is covered (e.g., 50%, 75%, 90%).

Maximum VNA coverage of a structure can be selected for the programmer to automatically calculate which programmer settings would provide maximum VNA coverage of the target structure without regard to surrounding anatomy. This setting is advantageous when complete coverage of a target structure or a sub-region of the target structure is desired for stimulation therapy.

Maximum VNA coverage of a structure with no VNA outside of the structure can be selected if the clinician is primarily concerned with side effects that could occur from the VNA affecting structures adjacent to the target structure. This selection would provide the settings that provide the most coverage of the structure of interest without any of the VNA spilling outside that structure.

Maximum VNA of a structure with clinician choosing which structure/s to have no VNA allows the clinician to select a target structure that the clinician desires the most VNA coverage of, and one or more anatomical structures the clinician wants to avoid. Thus, this stimulation field criteria can provide the most coverage of the structure of interest while avoiding any spill of the VNA into the structure/s to avoid.

At 306, the stimulation settings would produce stimulation fields in relation to the patient-specific anatomy based on selected criteria are determined. In embodiments, stimulation settings used to determine stimulation field shapes include pulse width, stimulation amplitude, and which electrode combination, contacts, and level should be used. In some embodiments, frequency and current are considered.

In embodiments, a search, such as a binary search, is used to determine VNA activations based on a vector out of each lead.

Starting with no produced VNA, settings can be adjusted such that the produced VNA gradually increases in size. Upon each adjustment, a check can be completed to determine if the VNA fully covers the target structure or contacts a separate anatomical structure. Based on the selected criteria, weights can be applied to produced VNA to determine stimulation settings that should be suggested. For example, if a clinician selects maximum VNA coverage of a structure with no VNA outside of the structure, any produced VNA that contains VNA coverage outside of the target structure can be automatically discarded and further adjustment of the VNA can be stopped. Accordingly, dynamically minimalistic search and/or a systematically reductive search can be used to determine VNA according to embodiments.

At 308, the programming clinician is presented with the determined stimulation settings and/a visual representation of the produced VNA in relation to the patient-specific anatomy. In embodiments, this information is presented via the programmer UI.

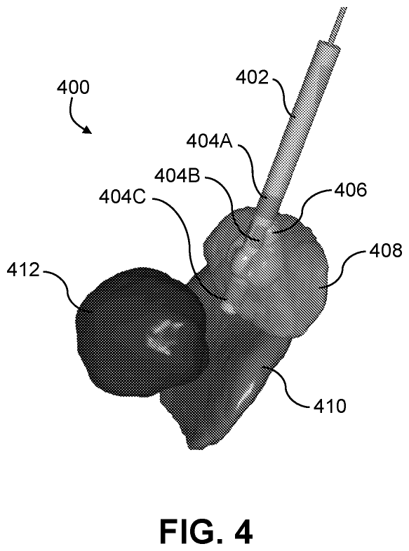

A perspective view of an electrode visualization 400 for a lead 402 producing VNA 406 in patient-specific anatomy including a target structure 408 and anatomical structures 410, 412 is depicted in FIG. 4 according to an embodiment. As can be seen, VNA 406 is visually represented overlapping a portion of target structure 408. A color key can be used to efficiently convey the target structure and overlapped regions. The distal end of lead 402 can include electrodes 404A, 404B, 404C.

The VNA displayed relative to the target structure allows the clinician to confirm that the combination is optimally delivering stimulation to the one or more target regions while avoiding stimulation of undesirable anatomical structures/sub-regions. By knowing the shape of the structure of interest and its location and orientation in relation to the implanted stimulation lead, the programming clinician can make an informed decision on the best stimulation settings for the patient.

In some embodiments, the VNA of the tissue activated with the stimulation electrode combination may be plotted via visual programming software, e.g., SureTune™.

Optionally at 310, the clinician could use the determined stimulation settings as the starting point for making adjustments based on their clinical judgement before starting stimulation. Otherwise, the clinician can simply use the suggested settings as-is and begin stimulation.

It should be understood that the individual operations used in the methods of the present teachings may be performed in any order and/or simultaneously, as long as the teaching remains operable. For example, the operations of method 300 could occur such that VNA criteria is received prior to patient-specific anatomy data. Furthermore, it should be appreciated that the apparatus and methods of the present teachings can include any number, or all, of the described embodiments, as long as the teaching remains operable.

In embodiments, the VNA corresponding to each of the stimulation field criteria can be found in advance of presenting the clinician with the selectable settings. In such embodiments, the clinician can preview the visual representations of each VNA situated within the patient-specific anatomy before selecting a setting.

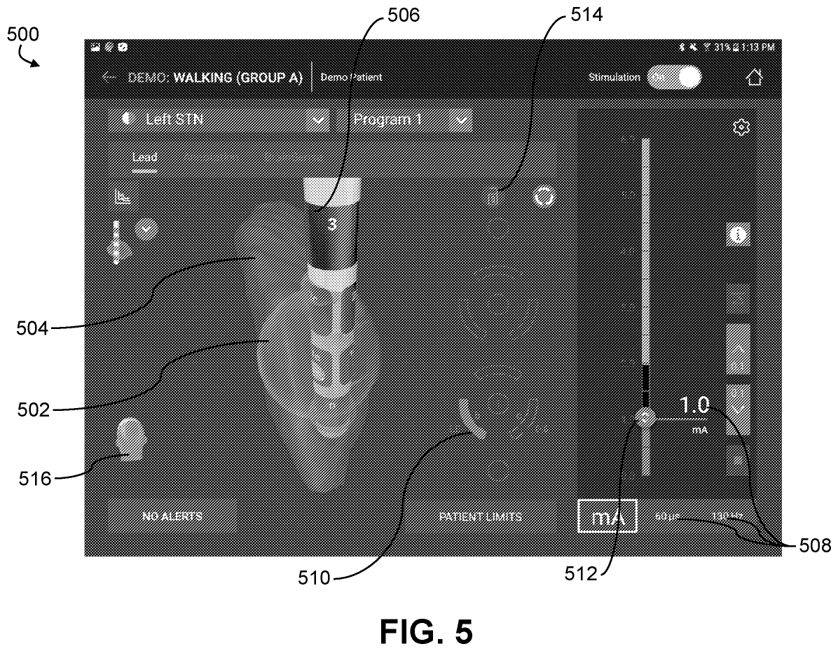

Referring to FIG. 5, a UI 500 configured to present VNA 502 oriented with respect to patient anatomy 504 and distal end of lead 506 is depicted according to an embodiment. UI 500 presents the DBS program settings 508 including electrode settings 510 that produce the depicted VNA. Slider 512 enables the clinician to easily select and edit program settings 508. Lock button 514 can be used to lock the shape of the VNA when interacting with UI 500. Lock button accordingly allows for slider 512 to be used to adjust the size of the VNA shape proportionally. View orientation 516 indicates the orientation of the based on the view and angle of patient anatomy 504 within UI 500.

Embodiments of the present disclosure provide for a simplified selection process for initial treatment VNA, reducing the time clinicians must spend in a programming session, whether intraoperatively, which reduces risk to the patient by reducing procedure time, or at a follow-up session, allowing the clinician to focus on other aspects of the patient's care or see more patients. Embodiments of the present disclosure could also potentially lead to better outcomes by providing the most coverage of the structure of interest, and/or avoiding structures that may cause unwanted side effects. Additionally, embodiments of the present disclosure can lead to improved battery life of the IMD through optimization of the VNA to reduce unnecessary stimulation outside of target regions and at non-target regions.

Embodiments described herein can be utilized for initial programming of a medical device. In further embodiments, subsequent or on-going programming can further be conducted. For example, after an initial brain sense survey, a subsequent brain sense survey can be utilized to adjust the VNA of the medical device. The subsequent brain sense survey can integrate specific patient data to reflect disease progression or other states after initial programming.

In an embodiment, various machine learning algorithms can be utilized. For example, in an embodiment to predict stimulation settings, machine learning algorithms can be applied to a preclinical dataset. Machine learning algorithms can be applied to selecting the VNA that is aligned with one or more of the predefined criteria. In another embodiment, the historical patient data can be utilized in combination with patient-specific information to suggest a pre-defined criteria for the clinician. Thus, patient-specific data can be compared to other patient data based on identified neurological disorders, including Parkinson's Disease, essential tremor, dystonia, and epilepsy to suggest a VNA criteria.

In embodiments one or more machine learning models can be built using data from a patient data pool. For example, embodiments can utilize a database of patient data. Models can be generated that for example, discriminate by neurological disorders or patient-anatomy characteristics and relative lead location. The more alike certain patient data is, the easier algorithms are able to predict.

Such programmer setting selection can be accomplished by computing similarity metrics for past patients using correlation or machine learning regression algorithms. For example, if the similarity of a VNA produced based on stimulation field criteria to training data of VNAs previously selected to address symptoms of the neurological disorder is above a certain threshold, (e.g., 75%, 90%, 95% or 99% similarity) a matching process can determine that the VNA represents programmer settings likely to address underlying symptoms. These predicted VNA can be presented to the clinician as selectable settings during a programming session.

In another embodiment, feedback can be used to improve recommendations to clinicians regarding anatomical structures to avoid or target with stimulation. For example, if machine learning predicts using a particular VNA edited by a clinician is no longer in place to stimulate a region of interest or overlaps a structure known to cause side-effects, a message or alert can be provided to physician (not to stimulate there) as a safety feature.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

The following are illustrative of the techniques described herein.

Example 1: A system for therapy program selection for a patient having an implanted medical device, comprising: a programmer device including computing hardware of at least one processor and memory operably coupled to the at least one processor; and instructions that, when executed on the programmer device, cause the programmer device to: receive patient-specific anatomy data including a location and an orientation for an implanted lead relative to one or more anatomical structures, wherein the one or more anatomical structures include a target structure; receive an indication of stimulation field criteria; determine stimulation settings based on the patient-specific anatomy data; display, via a user interface of the programmer device, a visualization of a Volume of Neural Activation (VNA) produced by the determined stimulation settings within the patient-specific anatomy data.

Example 2: The system of Example 1, wherein the indication of stimulation field criteria is one of maximum VNA coverage of the target structure, maximum VNA coverage of the target structure with minimal VNA outside of the target structure, and maximum VNA of the target structure with no VNA in one of the one or more anatomical structures.

Example 3: The system of Example 2, wherein prior to receiving an indication of stimulation field criteria the programmer device is further configured to: display, via the user interface, a prompt to select one of the stimulation field criteria Example 4: The system of Example 1, wherein the programmer device is further configured to: receive, via the user interface, an indication that one of the one or more anatomical structures should not be covered by the VNA.

Example 5: The system of Example 1, wherein the stimulation settings include one or more of electrode contacts used, pulse width, and stimulation amplitude.

Example 6: The system of Example 1, wherein a binary search is used to determine the stimulation settings.

Example 7: The system of Example 1, wherein the programmer device is further configured to: receive, via the user interface, instructions to adjust the determined stimulation settings.

Example 8: The system of Example 7, wherein the programmer device is further configured to: update the visualization based on the received instructions.

Example 9: The system of Example 1, wherein the programmer device is further configured to: program the implanted medical device to deliver electrical stimulation via the implanted lead based on the determined stimulation settings.

Example 10: The system of Example 1, wherein the determined stimulation settings can be selectively locked.

Example 11: A method for therapy program selection for a patient having an implanted medical device, comprising: receiving patient-specific anatomy data including a location and an orientation for an implanted lead relative to one or more anatomical structures, wherein the one or more anatomical structures include a target structure; receiving an indication of stimulation field criteria; determining stimulation settings based on the patient-specific anatomy data; displaying a visualization of a Volume of Neural Activation (VNA) produced by the determined stimulation settings within the patient-specific anatomy data.

Example 12: The method of Example 11, wherein the indication of stimulation field criteria is one of maximum VNA coverage of the target structure, maximum VNA coverage of the target structure with minimal VNA outside of the target structure, and maximum VNA of the target structure with no VNA in one of the one or more anatomical structures.

Example 13: The method of Example 12, further comprising, prior to receiving an indication of stimulation field: displaying a prompt to select one of the stimulation field criteria.

Example 14: The method of Example 11, further comprising: receiving an indication that one of the one or more anatomical structures should not be covered by the VNA.

Example 15: The method of Example 11, wherein the stimulation settings include one or more of electrode contacts used, pulse width, and stimulation amplitude.

Example 16: The method of Example 11, wherein determining stimulation settings is based on a binary search.

Example 17: The method of Example 11, further comprising: receiving instructions to adjust the determined stimulation settings.

Example 18: The method of Example 17, further comprising: updating the visualization based on the received instructions.

Example 19: The method of Example 11, further comprising: programming the implanted medical device to deliver electrical stimulation via the implanted lead based on the determined stimulation settings.

Example 20: The method of Example 11, wherein the determined stimulation settings can be selectively locked.

1. A system for therapy program selection for a patient having an implanted medical device, comprising:
a programmer device including computing hardware of at least one processor and memory operably coupled to the at least one processor; and
instructions that, when executed on the programmer device, cause the programmer device to:
receive patient-specific anatomy data including a location and an orientation for an implanted lead relative to one or more anatomical structures, wherein the one or more anatomical structures include a target structure;
receive an indication of stimulation field criteria;
determine stimulation settings based on the patient-specific anatomy data;
display, via a user interface of the programmer device, a visualization of a Volume of Neural Activation (VNA) produced by the determined stimulation settings within the patient-specific anatomy data.

2. The system of claim 1, wherein the indication of stimulation field criteria is one of maximum VNA coverage of the target structure, maximum VNA coverage of the target structure with minimal VNA outside of the target structure, and maximum VNA of the target structure with no VNA in one of the one or more anatomical structures.

3. The system of claim 2, wherein prior to receiving an indication of stimulation field criteria the programmer device is further configured to:
display, via the user interface, a prompt to select one of the stimulation field criteria.

4. The system of claim 1, wherein the programmer device is further configured to:
receive, via the user interface, an indication that one of the one or more anatomical structures should not be covered by the VNA.

5. The system of claim 1, wherein the stimulation settings include one or more of electrode contacts used, pulse width, and stimulation amplitude.

6. The system of claim 1, wherein a binary search is used to determine the stimulation settings.

7. The system of claim 1, wherein the programmer device is further configured to:
receive, via the user interface, instructions to adjust the determined stimulation settings.

8. The system of claim 1, wherein the programmer device is further configured to:
program the implanted medical device to deliver electrical stimulation via the implanted lead based on the determined stimulation settings.

9. The system of claim 1, wherein the determined stimulation settings can be selectively locked.

10. A method for therapy program selection for a patient having an implanted medical device, comprising:
receiving patient-specific anatomy data including a location and an orientation for an implanted lead relative to one or more anatomical structures, wherein the one or more anatomical structures include a target structure;
receiving an indication of stimulation field criteria;
determining stimulation settings based on the patient-specific anatomy data;
displaying a visualization of a Volume of Neural Activation (VNA) produced by the determined stimulation settings within the patient-specific anatomy data.

11. The method of claim 10, wherein the indication of stimulation field criteria is one of maximum VNA coverage of the target structure, maximum VNA coverage of the target structure with minimal VNA outside of the target structure, and maximum VNA of the target structure with no VNA in one of the one or more anatomical structures.

12. The method of claim 10, further comprising:
receiving an indication that one of the one or more anatomical structures should not be covered by the VNA.

13. The method of claim 11, further comprising:
receiving instructions to adjust the determined stimulation settings.

14. The method of claim 11, further comprising:
programming the implanted medical device to deliver electrical stimulation via the implanted lead based on the determined stimulation settings.

15. The method of claim 11, wherein the determined stimulation settings can be selectively locked.

16. The system of claim 7, wherein the programmer device is further configured to:
update the visualization based on the received instructions.

17. The method of claim 10, wherein the stimulation settings include one or more of electrode contacts used, pulse width, and stimulation amplitude.

18. The method of claim 10, wherein determining stimulation settings is based on a binary search

19. The method of claim 11, further comprising, prior to receiving an indication of stimulation field:
displaying a prompt to select one of the stimulation field criteria.

20. The method of claim 13, further comprising:
updating the visualization based on the received instructions.
